(19) United States
(12) Patent Application Publication
(10) Pub. No.: US 2008/0293821 A1
(43) Pub. Date: Nov. 27, 2008

(54) Cooling compositions

(76) Inventors: Ashok K. Moza, Baytown, TX (US); Hong Sun, Baytown, TX (US); Stephen Rohde, Baytown, TX (US); Diane Leffingwell, Baytown, TX (US); John Charles Leffingwell, Baytown, TX (US)

Correspondence Address: Qaroma Inc.;Subsidary of Chemicals Incorporated, 12321 Hatcherville Road, Baytown, TX, 77521, US

(21) Appl. No.: 11/804,760

(22) Filed: May 21, 2007

(51) Int. Cl. A61K 31/16; A23G 4/08; A23L 1/48

(52) U.S. Cl. 514/629; 426/3; 426/660

(57) ABSTRACT

Disclosed are improved cooling compositions comprised of N-(2-hydroxyethyl)-2,3-dimethyl-2-isopropylbutyramide and at least one other cooling agent. The cooling compositions may be used alone or in products such as a chewing gum or a confection.

### FIELD

This description relates to improved cooling compositions that may be delivered orally or to the skin or mucous membranes. The compositions contain one or more cooling agents in combination with N-(2-hydroxyethyl)-2,3-dimethyl-2-isopropylbutyramide. The cooling agent(s) and N-(2-hydroxyethyl)-2,3-dimethyl-2-isopropylbutyramide may be provided in one composition (for example, as liquid eutectic mixtures) or they may be provided separately in distinct formulations.

### BACKGROUND OF THE INVENTION

A number of substances are known to provide a sensation of cooling on application and are called “cooling agents.” Examples of cooling agents include menthol, (−)-(3S,3aS,3bR,4S,7R,7aR)-4-isopropyl-3,7-dimethyloctahydro-1H-cyclopenta[1,3]cyclopropa[1,2]benzen-3-ol (Cubebol), isopulegol, 3-(1-menthoxy)propan-1,2-diol, p-menthan-3,8-diol, 6-isopropyl-9-methyl-1,4-dioxaspiro-(4,5)-decane-2-methanol (Frescolat MGA), menthyl lactate (Frescolat ML), menthyl succinate, alkaline earth salts of menthyl succinate, 3,5,5-trimethylcyclohexanol, Ethyl N-[(5-methyl-2isopropylcyclohexyl)carbonyl]glycinate (WS-5), menthol propylene glycol carbonate (Frescolat MPC), monomenthyl glutarate, 2-{[(1R,2S,5R)-2-isopropyl-5-methylcyclohexyl]oxy}ethanol, menthyl pyrrolidone carboxylate (Questice), N,N-Dimethyl menthyl succinamide, Menthyl 3-hydroxybutyrate, Menthol ethylene glycol carbonate (Frescolat MGC), (1R,3R,4S)-3-menthyl 3,6-dioxaheptanoate, (1R,2S,5R)-3-menthyl methoxyacetate, (1R,2S,5R)-3-menthyl 3,6,9-trioxadecanoate, (1R,2S,5R)-3-menthyl 3,6,9-trioxadecanoate, (1R,2S,5R)-3-menthyl (2-hydroxyethoxy)acetate, (1R,2S,5R)-menthyl 11-hydroxy-3,6,9-trioxaundecanoate, 1-[2-hydroxyphenyl]-4-[2-nitrophen]1,2,3,6-tetrahydropyrimidine-2-one (Icilin, also known as AG-3-5), 3-[(2-isopropyl-5-methylcyclohexyl)oxy]-2-methylpropane-1,2-diol, N-(2-ethoxyethyl)-2,3-dimethyl-2-isopropylbutyramide, N-(3-methoxypropyl)-2,3-dimethyl-2-isopropylbutyramide, N-(3-ethoxypropyl)-2,3-dimethyl-2-isopropylbutyramide, N-(3-isopropoxypropyl)-2,3-dimethyl-2-isopropylbutyramide, N-(3-butoxypropyl)-2,3-dimethyl-2-isopropylbutyramide, 2-(5′-methyl-2′-(methylethyl)cyclohexyloxy)ethan-1-ol, 3-(5′-methyl-2′-(methylethyl)cyclohexyloxy)propan-1-ol, 4-(5′-methyl-2′-(methylethyl)cyclohexyloxy)butan-1-ol, menthol, peppermint oil, cornmint oil, menthone & eucalyptol.

Cooling agents are well known in the art and are described in the review by Leffingwell, “Cool without Menthol & Cooler than Menthol and Cooling Compounds as Insect Repellents,” (http://www.leffingwell.com/cooler_than_menthol.htm) and in the article by Mark Erman, Progress in Physiological Cooling Agents, Perfumer & Flavorist, Vol. 29, No. 8, pp. 34-50, the disclosures of which are herein incorporated by reference.

Despite the existence of these known cooling agents, a need still exists for cooling compositions that contribute a pleasant long-lasting cooling sensation to products without imparting unwanted off-flavor characteristics or undesirable aftertaste. Such new cooling compositions are desirable to provide a clean, high-quality flavor for confections and chewing gum. The use of N-(2-hydroxyethyl)-2,3-dimethyl-2-isopropylbutyramide (in combination with other cooling agents) in food, cosmetic and fragrance applications offers the opportunity to provide cooling compositions which provide strong and substantive refreshing and cooling attributes in the absence of negative flavor, aroma and cooling attributes.

The action of menthol and various non-menthol coolant compounds on “thermoreceptors” provides the “cool” sensation via cold receptors. In the case of menthol and certain other coolant compounds one can also get a “hot”, harsh or stinging “pain” sensation. Menthol can act at high concentrations in much the same way as capsaicin to produce a hot sensation, but in this case, it stimulates the fibers that register both cold temperatures as well as those that respond to warmth. Recently (2001), Gordon Reid & Maria-Luiza Flonta at the University of Bucharest have discovered an inward ionic current that is activated by moderate cooling in a small number of rat sensory neurons. This current has features that are found in intact cold receptors, including sensitization by menthol, adaptation upon sustained cooling, and modulation by calcium, and is likely to be important in cold sensing. Early models indicated that menthol stimulates cold receptors by blocking voltage-dependent Ca2+ channels, leading to a reduction in intracellular Ca2+ and inhibition of Ca2+-dependent K+ channels. However, Reid and others have since discovered that menthol stimulates entry of Ca2+ and increases intracellular Ca2+ concentration in cold-sensitive neurons; thus stimulation of cold receptors by menthol can be explained more simply by sensitization of the cold-induced inward Ca2+ current. See Reid, G., Flonta M-L., Cold current in thermoreceptive neurons, Nature, 413, 480 (2001).

In the Mar. 7, 2002 issue of Nature, McKemy, Neuhausser & Julius have characterized and cloned a menthol receptor from trigeminal sensory neurons that is also activated by thermal stimuli in the cool to cold range. This cold- and menthol-sensitive receptor, CMR1, is a member of the TRP family of excitatory ion channels, and they propose that it functions as a transducer of cold stimuli in the somatosensory system. These findings, together with their previous identification of the heat-sensitive channels VR1 and VRL-1, demonstrate that TRP channels detect temperatures over a wide range and are the principal sensors of thermal stimuli in the mammalian peripheral nervous system. See David D. McKemy, Werner M. Neuhausser, David Julius, Identification of a cold receptor reveals a general role for TRP channels in thermosensation, Nature 416, 52-58 (2002).

In the same issue of Nature, Charles Zucker explains how the discovery of a cold-sensitive ion channel will help dissect how the nervous system encodes and decodes the temperature spectrum. See Nature, 416, 27-28 (2002)

Similarly, Andrea Peier, et. al., described the cloning and characterization of TRPM8, a receptor activated by cold temperatures and by the cooling agent, menthol. See Cell, 108 (5), 705-15 (2002)

Also in March 2002, the work of Viana & co-workers suggests that cold sensitivity is not associated to a specific transduction molecule but instead results from a favorable blend of ionic channels expressed in a small subset of sensory neurons. See Nat. Neurosci., March 5 (3):254-60 (2002) and J. Neurosci., 26(48):12512-25 (2006)

In 2003, Patapoutian, et. al., reviewed the status of the mechanisms of temperature sensations. See Nature Reviews Neuroscience, 4, 529-539 (2003)

In February 2004, H-J Behrendt, et. al., published a study on the effects of 70 odorants and menthol-related substances on recombinant cold-menthol receptor TRPM8 (mTRPM8), expressed in HEK293 cells. These were examined using a fluorometric imaging plate reader (FLIPR®) assay. In all, 10 substances (linalool, geraniol, hydroxycitronellal, WS-3, WS-23, Frescolat MGA, Frescolat ML, PMD38, Coolact P and Cooling Agent 10) were found to be agonists. A summary of the potencies and efficacies were shown. This work gives a new approach for screening cooling compounds. See British Journal of Pharmacology 141, 737-745 (2004)

### OBJECTS OF THE INVENTION

An object of the invention is to provide improved cooling compositions that produce a pleasant prolonged physiological cooling sensation to the skin or mucous membranes. The cooling compositions may be present alone or in products such chewing gums or confections for providing a pleasant long lasting cooling effect, often with improved flavor characteristics, over the use of a single cooling agent alone.

Another object of the invention is to disclose the utility of compositions containing one or more cooling agents in combination with N-(2-hydroxyethyl)-2,3-dimethyl-2-isopropylbutyramide. The cooling agent(s) and N-(2-hydroxyethyl)-2,3-dimethyl-2-isopropylbutyramide may be provided in one composition (for example, as liquid eutectic mixtures) or they may be provided separately in distinct formulations.

### SUMMARY OF THE INVENTION

The cooling compositions described herein provide a pleasant prolonged physiological cooling sensation to the skin or mucous membranes. The cooling compositions may be present alone or in products such chewing gums or confections for providing a pleasant long lasting cooling effect, often with improved flavor characteristics, over the use of a single cooling agent alone.

Such cooling compositions may provide for delivering a sensation similar to that delivered by menthol, N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5) alone by administering a composition containing N-(2-hydroxyethyl)-2,3-dimethyl-2-isopropylbutyramide but where the amount of menthol, N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5) present in the composition can be reduced relative to the amount of menthol, N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5) alone required to provide a substantially similar cooling sensation.

At high use levels certain cooling agents (e.g. menthol) provide a degree of “hotness” and “harshness” which limits the useful amount that may be employed.

In addition, we have found that employing N-(2-hydroxyethyl)-2,3-dimethyl-2-isopropylbutyramide in conjunction with a second cooling agent and optionally a third cooling agent comprised of either (−)-(1R,2R,5S)-2-isopropyl-5-methylcyclohexanol also more commonly known as laevo-menthol) or racemic (1RS,2RS,5SR)-2-isopropyl-5-methylcyclohexanol (racemic menthol) provides a pleasant long lasting cooling effect, often with improved flavor characteristics, over the use of a single cooling agent alone.

### DETAILED DESCRIPTION

This description extends to products such as flavoring agents, foodstuffs, confections, beverages, gums, dentifrices, mouthwashes, toiletries, liniments, lotions for topical application and cigarettes, such products comprising cooling compositions that provide a substantially cooling physiological sensation.

Generally, the compositions will contain an amount of the active cooling compounds sufficient to stimulate the cold receptors in the areas of the skin or mucous membrane with which the compositions come into contact and thereby promote the desired level of cold sensation. As the degree and longevity of cooling sensation varies from compound to compound the quantity of stimulant used in each composition may vary.

The synthesis of N-(2-hydroxyethyl)-2,3-dimethyl-2-isopropylbutyramide is described in U.S. Pat. No. 7,030,273 (Apr. 18, 2006) and its cooling properties are indicated only in a general way. No indication is provided that it will, in combination with other cooling agents, provide a pleasant long lasting cooling effect, often with improved flavor characteristics, over the use of a single cooling agent alone.

N-(2-hydroxyethyl)-2,3-dimethyl-2-isopropylbutyramide is sold under the trade name of “ICE 6000” by Qaroma, Inc., a division of Chemicals Incorporated.

Accordingly, we herein disclose that N-(2-hydroxyethyl)-2,3-dimethyl-2-isopropylbutyramide in combination with other cooling agents, provides a pleasant long lasting cooling effect, often with improved flavor characteristics in various applications.

In particular, in a preferred embodiment, it has been found that N-(2-hydroxyethyl)-2,3-dimethyl-2-isopropylbutyramide in combination with a second cooling agent selected from the group consisting of N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinat (WS-5) provides a pleasant long lasting cooling effect, often with improved flavor characteristics in various applications.

Further, in a preferred embodiment, it has been found that a cooling composition comprising a N-(2-hydroxyethyl)-2,3-dimethyl-2-isopropylbutyramide, menthol, and a third cooling agent selected from the group consisting of N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5) provides a pleasant long lasting cooling effect, often with improved flavor characteristics in various applications.

Further, in a preferred embodiment, N-(2-hydroxyethyl)-2,3-dimethyl-2-isopropylbutyramide is present in an amount of 5 to 70 weight percentage of the cooling composition and preferably in an amount of 15 to 70 weight percentage of the cooling composition.

The cooling compositions can be used in any products intended for oral, skin or mucosal delivery as a component for providing sensation of cooling and for providing a physiological effect that is substantially cooling. The products for which the compositions are useful include, but are not limited to, food and drink, such as candies, drops, chewing gums, tablets, chocolates, cakes, cookies, snack food, bread, tea, coffee, juice, fruit drinks, fruit wine, dairy drinks, carbonated beverages, alcoholic beverages and seasonings; and oral care preparations, such as mouthwash, toothpaste, nebulizers, drinks, medicinal drops, gargles, and chewables.

The following illustrates a more comprehensive range of products into which the active cooling composition can be incorporated. These include, for instance, edible or potable compositions including alcoholic and non-alcoholic beverages, confectionary, chewing gum; cachous; ice cream; jellies. These further include toiletries including after shave lotions, shaving soaps, creams and foams, toilet water, deodorants and antiperspirants, “solid colognes,” toilet soaps, bath oils and salts, shampoos, hair oils, talcum powders, face creams, hand creams, sunburn lotions, cleansing tissues, dentifrices, toothpicks, mouthwashes, hair tonics, eye drops. Additionally, the range extends to medicaments including antiseptic ointments, pile ointments, liniments, lotions, decongestants, counter-irritants, cough mixtures, throat lozenges, antacid and indigestion preparations, oral analgesics. Also contemplated are tobacco preparations including cigars, cigarettes, pipe tobacco, chewing tobacco and snuff and cigarette tobacco filters. Even further are contemplated miscellaneous compositions such as water soluble adhesive compositions for envelopes, postage stamps, adhesive labels etc.

The edible and potable compositions will contain the active cooling composition in combination with an edible carrier and usually a flavoring or coloring agent. In general, the cooling composition may be present in amounts in the range 0.0005 to 5% by weight based on the total composition. Similar considerations apply to the formulation of beverages. In general, the amount of the cooling composition used will generally be in the range 0.0005 to 1.0% by weight based on the total composition. Because of the cooling sensation imparted to the skin, the amount of the cooling composition added to toiletries will usually be in the range 0.1 to 8% by weight based on the total composition. Medicaments will normally feature an amount of the cooling composition of from 0.01 to 2.0% by weight. Tobacco preparations may contain as little as 0.1% of the composition.

In addition to the cooling composition described herein, these products can contain other additives according to use. For example, additives permitted by various countries Food Sanitation Laws can be added to food and drink according to necessity. Useful additives include saccharides, sweeteners, inorganic salts, emulsifiers, acidifiers, flavorings, colors, antioxidants, raising agents, thickeners, vegetable oils, milk, and other dairy products. In some detail, bakery products can comprise wheat flour (base), butter, a raising agent, e.g., baking powder, an emulsifier, e.g., a sucrose fatty acid ester, saccharides, e.g., sugar, inorganic salts, and flavorings. Chocolate can comprise cacao mass (base) cacao butter, saccharides, e.g., sugar, milk, and an emulsifier. Emulsified dressings can comprise salad oil, water, vinegar, sugar, thickening polysaccharides, and sweeteners. Chewing gum can comprise a gum base, saccharides, such as sugar, glucose, corn syrup and maltodextrins, and flavors. Candy can comprise saccharides, acidifiers, e.g., citric acid, sweeteners, flavorings, and colors. Orange fruit drinks can comprise orange juice, sweeteners, e.g., isomerized sugars, acidifiers, e.g., citric acid, and antioxidants, e.g., vitamin C. Other drinks can comprise fruit juices as well as dairy products such as milk and chocolate milk, which may contain saccharides, e.g., sugar, stabilizers, e.g., carboxymethylcellulose, acidifiers, e.g., citric acid, and flavorings, e.g., a strawberry flavor. In addition, the cooling compositions of the invention can be employed in bottled water to provide the sensation of “cold water” for products at ambient temperature.

In an embodiment, the cooling compositions are used in chewing gums. Some chewing gum formulations are described in, for instance, U.S. Pat. Nos. 6,627,233, 6,685,916 and 6,696,044, herein incorporated by reference. Additives which can be used in the preparations include inorganic salts, inorganic oxides, organic salts, thickeners, wetting agents, emulsifiers, surface active agents, humectants, alcohols, color additives, flavorings, and, if desired, medical ingredients such as crude drugs, hemostatics, circulation stimulants, anti-inflammatory agents, astringents, antibacterial and/or antifungal agents, and bactericides. In particular embodiments, toothpaste can comprise abrasives, such calcium phosphate, as calcium carbonate, aluminum hydroxide, silica, and calcium pyrophosphate; wetting agents, such as glycerin, sorbitol, and propylene glycol; tackifiers, such as carboxymethyl cellulose, carrageenan, and hydroxyethyl cellulose; surface active agents, such as sodium laurylsulfate, N-acylglutaminates, and sucrose fatty acid esters; sweeteners, such as sodium saccharin , aspartame, sucralose and xylitol.

In particular embodiments, the cooling compositions are used in chewing gums and confections. Compositions of chewing gum and confections are well known in the art and described in depth in, for instance, U.S. Pat. Nos. 6,685,916, 6,627,233, 6,685,916 and 6,696,044, the disclosures of which are incorporated by reference.

Chewing gum compositions may include one or more of gum bases, flavoring agents and bulk sweeteners. The term “confections” as used herein includes, but is not limited to: nougats, candies, panning goods, gel confections, fondants, lozenges, hard boiled candies, mints, troches, pastilles, microcapsules, and fast-dissolving solid forms including freeze dried forms (cakes, wafers, thin films, and tablets) and fast dissolving solid forms including compressed tablets. The term “fast dissolving solid form” as used herein means that the solid dosage form dissolves in less than about 60 seconds, preferably less than about 15 seconds, more preferably less than about 5 seconds, in the oral cavity. Lozenges include discoid shaped solids comprising a therapeutic agent in a flavored base. The base may be a hard sugar candy, gelatin, or a combination of sugar with sufficient mucilage to give it form. Lozenge compositions (compressed tablet type) typically include one or more fillers (compressible sugar), flavoring agents and lubricants.

The cooling compositions may be used in either regular chewing gum, pressed gum or bubble gum. Center filled gum is another common chewing gum form. The gum portion has a similar composition and mode of manufacture to that described above. However, the center fill is typically an aqueous solution or gel, which can be injected into the center of the gum during processing. The cooling agents and compositions may optionally be incorporated together or singly into the center fill during manufacture of the fill or into the chewing gum. The center fill gum may also be optionally coated and may be prepared in various forms such as in the form of a lollipop.

In accordance with one aspect of the chewing gum composition, the cooling compositions are added during the manufacture of the chewing gum composition, that is, with the sweeteners, flavoring agents and the like.

The present description also encompasses confectionary products containing a composition of individual agents that in total impart a physiological effect that can be substantially cooling. Confectionary compositions include compressed tablets such as mints, hard boiled candies, nougats, gels, center fill confections, fondants, panning goods and other compositions falling within the generally accepted definition of confectionary compositions.

Further details regarding the preparation of confectionary compositions can be found in Skuse's Complete Confectioner (13th Edition) (1957) including pp. 41-71, 133-144, and 255-262; and Sugar Confectionary Manufacture (2nd Edition) (1995), E. B. Jackson, Editor, pp. 129-168, 169-188, 189-216, 218-234, and 236-258, the content of which is incorporated herein by reference.

In the following examples, a panel consisting of both professional flavorists and other individuals with sensory testing experience was employed. In each tasting session samples were presented to the panelists in a randomized order to avoid presentation order effect. Panelists were asked to rate the samples as same or different. If different, panelists were asked to rate the cooling sensation relative to cooling onset time, duration, smoothness, and describe the overall flavor effect.

### EXAMPLE 1

Flavor Comparison of N-(2-hydroxyethyl)-2,3-dimethyl-2-isopropylbutyramide versus N-Ethyl-p-menthane-3-carboxamide (WS-3) in a confectionary base.

a) To a melted commercial candy confectionary base (Wilton Yellow Candy Melts®), was added 700 ppm (0.07%) of N-(2-hydroxyethyl)-2,3-dimethyl-2-isopropylbutyramide in the form of a 10% solution in alcohol. The resulting confection mixture was poured into a pastille mold to form a soft confection.

b) To a melted commercial candy confectionary base (Wilton Yellow Candy Melts®), was added 300 ppm (0.03%) of N-Ethyl-p-menthane-3-carboxamide (WS-3) in the form of a 10% solution in alcohol. The resulting confection mixture was poured into a pastille mold to form a soft confection.

Upon comparison, panelists noted that while the overall cooling sensation was similar, the two formulas of Example 1-a and Example 1-b easily be differentiated. The cooling onset time for Example 1-a was described as shorter than for Example 1-b, while the cooling duration was somewhat longer for Example 1-b. In addition, while Example 1-b gave more of a cooling sensation on the back of the tongue and throat, Example 1-a provided a flavor effect to the front and sides of the tongue not present in Example 1-b.

Candy Melts® is a registered trademark of Wilton Industries, Inc.

### EXAMPLE 2

Flavor Comparison of a mixture of N-(2-hydroxyethyl)-2,3-dimethyl-2-isopropylbutyramide and N-Ethyl-p-menthane-3-carboxamide (WS-3), in a confectionary base, to the formulas in Example 1.

a) To a melted commercial candy confectionary base (Wilton Yellow Candy Melts®), was added 500 ppm of a mixture comprised of 60 weight percent N-(2-hydroxyethyl)-2,3-dimethyl-2-isopropylbutyramide and 40 weight percent of N-Ethyl-p-menthane-3-carboxamide (WS-3) in the form of a 10% solution in alcohol. The resulting confection mixture was poured into a pastille mold to form a soft confection.

Panelists describe the cooling sensation of Example 2-a as having a rapid onset and very long lasting duration. The panelists universally agreed that Example 2-a had a superior flavor as compared to Example 1-a and Example 1-b. Example 2-a was described as having a somewhat higher cooling sensation (suggesting a possible synergistic cooling effect) and a smoother and longer lasting cooling sensation than either Example 1-a or Example 1-b.

### EXAMPLE 3

### Flavor Comparisons of Coolant Compositions in a Cinnamon Flavored Confection

a) A cinnamon flavor was prepared by mixing the components in Table 1.

TABLE 1

| Ingredient | % |
|---|---|
| Cassia Oil | 2.22 |
| Cinnamic Aldehyde | 7.78 |
| Ethyl Alcohol | 90.00 |
| | 100.00 |

The cinnamon flavor was utilized with 4 hours in order to preclude acetal formation that could affect negatively the flavor profile.

b) To a melted commercial candy confectionary base (Wilton Red Candy Melts®), was added 1.2% of the cinnamon flavor from Table 1 and 700 ppm (0.07%) of N-(2-hydroxyethyl)-2,3-dimethyl-2-isopropylbutyramide in the form of a 10% solution in alcohol. The resulting confection mixture was poured into a pastille mold to form a soft confection.

c) To a melted commercial candy confectionary base (Wilton Red Candy Melts®), was added 1.2% of the cinnamon flavor from Table 1 and 300 ppm (0.03%) of N-Ethyl-p-menthane-3-carboxamide (WS-3) in the form of a 10% solution in alcohol. The resulting confection mixture was poured into a pastille mold to form a soft confection.

d) To a melted commercial candy confectionary base (Wilton Red Candy Melts®), was added 1.2% of the cinnamon flavor from Table 1 and 500 ppm of a mixture comprised of 60 weight percent N-(2-hydroxyethyl)-2,3-dimethyl-2-isopropylbutyramide and 40 weight percent of N-Ethyl-p-menthane-3-carboxamide (WS-3) in the form of a 10% solution in alcohol. The resulting confection mixture was poured into a pastille mold to form a soft confection.

Panelists found the soft confection of Example 3-d to be superior to either Example 3-b or Example 3-c in that Example 3-d was described as having a somewhat higher cooling sensation (suggesting a possible synergistic cooling effect) and a smoother and longer lasting cooling sensation than either Example 3-b or Example 3-c. In addition, the cinnamon flavor notes were significantly enhanced.

### EXAMPLE 4

### Flavor Comparisons of Coolant Compositions in a Peppermint Flavored Confection

a) To a melted commercial candy confectionary base (Wilton Red Candy Melts®), was added 0.4% of twice distilled peppermint oil and 700 ppm (0.07%) of N-(2-hydroxyethyl)-2,3-dimethyl-2-isopropylbutyramide in the form of a 10% solution in alcohol. The resulting confection mixture was poured into a pastille mold to form a soft confection.

b) To a melted commercial candy confectionary base (Wilton Red Candy Melts®), was added 0.4% of twice distilled peppermint oil and 300 ppm (0.03%) of N-Ethyl-p-menthane-3-carboxamide (WS-3) in the form of a 10% solution in alcohol. The resulting confection mixture was poured into a pastille mold to form a soft confection.

c) To a melted commercial candy confectionary base (Wilton Red Candy Melts®), was added 0.4% of twice distilled peppermint oil and 500 ppm of a mixture comprised of 60 weight percent N-(2-hydroxyethyl)-2,3-dimethyl-2-isopropylbutyramide and 40 weight percent of N-Ethyl-p-menthane-3-carboxamide (WS-3) in the form of a 10% solution in alcohol. The resulting confection mixture was poured into a pastille mold to form a soft confection.

Panelists found the soft confection of Example 4-c to be superior to either Example 4-a or Example 4-b in that Example 4-c was described as having a somewhat higher cooling sensation (suggesting a possible synergistic cooling effect) and a smoother and longer lasting cooling sensation than either Example 4-a or Example 4-b. In addition, the peppermint impression was enhanced.

1. A cooling composition comprising N-(2-hydroxyethyl)-2,3-dimethyl-2-isopropylbutyramide and a second cooling agent.

2. A composition according to claim 1, wherein the second cooling agent is selected from the group consisting of N-Ethyl-p-menthane-3-carboxamide (WS-3), 2-Isopropyl-N,2,3-trimethylbutyramide (WS-23), Ethyl N-[(5-methyl-2isopropylcyclohexyl)carbonyl]glycinate (WS-5) or menthol.

3. A cooling composition comprising N-(2-hydroxyethyl)-2,3-dimethyl-2-isopropylbutyramide, menthol, and a third cooling agent.

4. A composition according to claim 3, wherein the third cooling agent is selected from the group consisting of N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5)

5. A composition according to claim 1, wherein the N-(2-hydroxyethyl)-2,3-dimethyl-2-isopropylbutyramide is present in an amount of at least 5 weight percentage of the composition.

6. A composition according to claim 3, wherein the N-(2-hydroxyethyl)-2,3-dimethyl-2-isopropylbutyramide is present in an amount of at least 5 weight percentage of the composition.

7. A composition according to claim 1, wherein the N-(2-hydroxyethyl)-2,3-dimethyl-2-isopropylbutyramide is present in an amount of at least 10 weight percentage of the composition.

8. A composition according to claim 3, wherein the N-(2-hydroxyethyl)-2,3-dimethyl-2-isopropylbutyramide is present in an amount of at least 10 weight percentage of the composition.

9. A composition according to claim 1, wherein the N-(2-hydroxyethyl)-2,3-dimethyl-2-isopropylbutyramide is present in an amount of at least 20 weight percentage of the composition.

10. A composition according to claim 3, wherein the N-(2-hydroxyethyl)-2,3-dimethyl-2-isopropylbutyramide is present in an amount of at least 20 weight percentage of the composition.

11. A composition according to claim 1, wherein the N-(2-hydroxyethyl)-2,3-dimethyl-2-isopropylbutyramide is present in an amount of at least 30 weight percentage of the composition.

12. A composition according to claim 3, wherein the N-(2-hydroxyethyl)-2,3-dimethyl-2-isopropylbutyramide is present in an amount of at least 30 weight percentage of the composition.

13. A composition according to claim 1, wherein the N-(2-hydroxyethyl)-2,3-dimethyl-2-isopropylbutyramide is present in an amount of at least 40 weight percentage of the composition.

14. A composition according to claim 3, wherein the N-(2-hydroxyethyl)-2,3-dimethyl-2-isopropylbutyramide is present in an amount of at least 40 weight percentage of the composition.

15. A composition according to claim 1, wherein the N-(2-hydroxyethyl)-2,3-dimethyl-2-isopropylbutyramide is present in an amount of at least 50 weight percentage of the composition.

16. A composition according to claim 3, wherein the N-(2-hydroxyethyl)-2,3-dimethyl-2-isopropylbutyramide is present in an amount of at least 50 weight percentage of the composition.

17. A composition according to claim 1, wherein the N-(2-hydroxyethyl)-2,3-dimethyl-2-isopropylbutyramide is present in an amount of at least 60 weight percentage of the composition.

18. A composition according to claim 3, wherein the N-(2-hydroxyethyl)-2,3-dimethyl-2-isopropylbutyramide is present in an amount of at least 60 weight percentage of the composition.

19. A composition according to claim 1, wherein the N-(2-hydroxyethyl)-2,3-dimethyl-2-isopropylbutyramide is present in an amount of at least 70 weight percentage of the composition.

20. A composition according to claim 3, wherein the N-(2-hydroxyethyl)-2,3-dimethyl-2-isopropylbutyramide is present in an amount of at least 70 weight percentage of the composition.

21. A composition according to claim 1, wherein the N-(2-hydroxyethyl)-2,3-dimethyl-2-isopropylbutyramide is present in an amount of 15 to 70 weight percentage of the composition.

22. A composition according to claim 3, wherein the N-(2-hydroxyethyl)-2,3-dimethyl-2-isopropylbutyramide is present in an amount of 15 to 70 weight percentage of the composition.

23. A confection having a cooling composition comprising N-(2-hydroxyethyl)-2,3-dimethyl-2-isopropylbutyramide and a second cooling agent.

24. A confection according to claim 23, wherein the cooling composition further comprises menthol.

25. A confection according to claim 23, wherein the second cooling agent is selected from the group consisting of N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5).

26. A confection according to claim 23, wherein the N-(2-hydroxyethyl)-2,3-dimethyl-2-isopropylbutyramide is present in an amount of at least 5 weight percentage of the cooling composition.

27. A confection according to claim 23, wherein the N-(2-hydroxyethyl)-2,3-dimethyl-2-isopropylbutyramide is present in an amount of at least 10 weight percentage of the cooling composition.

28. A confection according to claim 23, wherein the N-(2-hydroxyethyl)-2,3-dimethyl-2-isopropylbutyramide is present in an amount of at least 20 weight percentage of the cooling composition.

29. A confection according to claim 23, wherein the N-(2-hydroxyethyl)-2,3-dimethyl-2-isopropylbutyramide is present in an amount of at least 30 weight percentage of the cooling composition.

30. A confection according to claim 23, wherein the N-(2-hydroxyethyl)-2,3-dimethyl-2-isopropylbutyramide is present in an amount of at least 40 weight percentage of the cooling composition.

31. A confection according to claim 23, wherein the N-(2-hydroxyethyl)-2,3-dimethyl-2-isopropylbutyramide is present in an amount of at least 50 weight percentage of the cooling composition.

32. A confection according to claim 23, wherein the N-(2-hydroxyethyl)-2,3-dimethyl-2-isopropylbutyramide is present in an amount of at least 60 weight percentage of the cooling composition.

33. A confection according to claim 23, wherein the N-(2-hydroxyethyl)-2,3-dimethyl-2-isopropylbutyramide is present in an amount of at least 70 weight percentage of the cooling composition.

34. A confection according to claim 23, wherein the N-(2-hydroxyethyl)-2,3-dimethyl-2-isopropylbutyramide is present in an amount of 15 to 70 weight percentage of the cooling composition.

35. A confection according to claim 23, wherein the N-(2-hydroxyethyl)-2,3-dimethyl-2-isopropylbutyramide is present in an amount of 0.0005 to 1.0 weight percentage of the confection.

36. A chewing gum having a cooling composition comprising N-(2-hydroxyethyl)-2,3-dimethyl-2-isopropylbutyramide and a second cooling agent.

37. A chewing gum according to claim 36, wherein the cooling composition further comprises menthol.

38. A chewing gum according to claim 36, wherein the second cooling agent is selected from the group consisting of N-Ethyl-p-menthane-3-carboxamide (WS-3), 2-Isopropyl-N,2,3-trimethylbutyramide (WS-23), or Ethyl N-[(5-methyl-2isopropylcyclohexyl)carbonyl]glycinate (WS-5).

39. A chewing gum according to claim 36, wherein the N-(2-hydroxyethyl)-2,3-dimethyl-2-isopropylbutyramide is present in an amount of at least 5 weight percentage of the cooling composition.

40. A chewing gum according to claim 36, wherein the N-(2-hydroxyethyl)-2,3-dimethyl-2-isopropylbutyramide is present in an amount of at least 10 weight percentage of the cooling composition.

41. A chewing gum according to claim 36, wherein the N-(2-hydroxyethyl)-2,3-dimethyl-2-isopropylbutyramide is present in an amount of at least 20 weight percentage of the cooling composition.

42. A chewing gum according to claim 36, wherein the N-(2-hydroxyethyl)-2,3-dimethyl-2-isopropylbutyramide is present in an amount of at least 30 weight percentage of the cooling composition.

43. A chewing gum according to claim 36, wherein the N-(2-hydroxyethyl)-2,3-dimethyl-2-isopropylbutyramide is present in an amount of at least 40 weight percentage of the cooling composition.

44. A chewing gum according to claim 36, wherein the N-(2-hydroxyethyl)-2,3-dimethyl-2-isopropylbutyramide is present in an amount of at least 50 weight percentage of the cooling composition.

45. A chewing gum according to claim 36, wherein the N-(2-hydroxyethyl)-2,3-dimethyl-2-isopropylbutyramide is present in an amount of at least 60 weight percentage of the cooling composition.

46. A chewing gum according to claim 36, wherein the N-(2-hydroxyethyl)-2,3-dimethyl-2-isopropylbutyramide is present in an amount of at least 70 weight percentage of the cooling composition.

47. A chewing gum according to claim 36, wherein the N-(2-hydroxyethyl)-2,3-dimethyl-2-isopropylbutyramide is present in an amount of 15 to 70 weight percentage of the cooling composition.

48. A chewing gum according to claim 36, wherein the N-(2-hydroxyethyl)-2,3-dimethyl-2-isopropylbutyramide is present in an amount of 0.005 to 1.2 weight percentage of the chewing.

49. A method for delivering a prolonged physiological cooling sensation to the skin or a mucous membrane comprising administering a composition according to claim 1.

50. A method for delivering a prolonged physiological cooling sensation to the skin or a mucous membrane comprising administering a composition according to claim 3.

51. A method for delivering a prolonged physiological cooling sensation to the skin or a mucous membrane comprising administering a composition according to claim 1 wherein the N-(2-hydroxyethyl)-2,3-dimethyl-2-isopropylbutyramide is admixed with N-Ethyl-p-menthane-3-carboxamide (WS-3) in the form of a liquid eutectic mixture.

52. A method for delivering a prolonged physiological cooling sensation to the skin or a mucous membrane comprising administering a composition according to claim 3 wherein the N-(2-hydroxyethyl)-2,3-dimethyl-2-isopropylbutyramide is admixed with N-Ethyl-p-menthane-3-carboxamide (WS-3) in the form of a liquid eutectic mixture.

53. A method for delivering a prolonged physiological cooling sensation to the skin or a mucous membrane comprising administering a composition according to claim 1 wherein the product consists of flavoring agents, foodstuffs, confections, beverages, gums, dentifrices, mouthwashes, toiletries, liniments, lotions for topical application and cigarettes, such products comprising cooling compositions that provide a substantially cooling physiological sensation.

54. A method for delivering a prolonged physiological cooling sensation to the skin or a mucous membrane comprising administering a composition according to claim 3 wherein the product consists of flavoring agents, foodstuffs, confections, beverages, gums, dentifrices, mouthwashes, toiletries, liniments, lotions for topical application and cigarettes, such products comprising cooling compositions that provide a substantially cooling physiological sensation.
